(19) United States
(12) Patent Application Publication
(10) Pub. No.: US 2024/0252821 A1
(43) Pub. Date: Aug. 1, 2024

(54) MUSCLE CONTRACTION CONTROL TO MANAGE DVT

(71) Applicant: RG Medical Technologies, Inc., Wixom, MI (US)

(72) Inventors: Ronald G. LeTourneau, Wixon, MI (US); Gregory W. Auner, Wixom, MI (US)

(73) Assignee: RG Medical Technologies, Inc., Wixom, MI (US)

(21) Appl. No.: 18/160,537

(22) Filed: Jan. 27, 2023

Related U.S. Application Data
(60) Provisional application No. 63/304,816, filed on Jan. 31, 2022.

(51) Int. Cl. A61N 1/36; A61N 1/04

(52) U.S. Cl. CPC A61N 1/36031; A61N 1/0452

(57) ABSTRACT

A device includes a conductive pad, an electrode in electrical communication with the conductive pad, and a controller. The controller is programmed to provide a plurality of temporally successive electrical signals to the electrode; determine a maximum amount of muscle contraction in response to the provided electrical signals based on sensor data; and select an optimized electrical signal by determining a minimum voltage that corresponds to the maximum amount of muscle contraction.

### CROSS-REFERENCE TO RELATED APPLICATION

This application claims priority to provisional U.S. Patent Appl. No. 63/304,816, filed on Jan. 31, 2022, which is hereby incorporated by reference in its entirety.

### BACKGROUND

A person may be non-ambulatory, e.g., while recovering from a surgical procedure. In these situations, the person may be relatively inactive, e.g., remaining in a lying position, for extended periods of time. Surgical procedures and/or extended periods of inactivity can cause venous thromboembolism (VT), i.e., blood clots in veins, including deep venous thrombosis (DVT), i.e., blood clots in a vein deep within the person's body, e.g., in a leg or pelvis. Other factors associated with the person's medical history, e.g., injuries, illness, age, weight, etc., can also cause VT and DVT. DVT can cause serious complications for the person. For example, DVT can cause damage to the valves in the vein, i.e., post-thrombotic syndrome, thereby causing the person to experience swelling, pain, and, in severe cases, ulcers. Another serious complication of DVT can happen if a part of the blood clot breaks off and travels to the lungs, via the bloodstream. Once in the lungs, the clot can block blood flow causing a pulmonary embolism (PE), which can lead to subsequent medical procedures (thereby increasing a recovery and/or non-ambulatory period) or death. Accordingly, it is desirable for a person to walk periodically following a surgical procedure; however, the person may be unable to walk for a period of time, e.g., while recovering, as a result of the surgical procedure.

Compression devices, e.g., sequential compression devices (SCD) or Intermittent Pneumatic Compression Devices (IPC), and conductive pads are used in the medical field, e.g., following surgical procedures, as a prophylaxis for DVT. Typically, compression devices include sleeves that encircle a portion of the person's leg, e.g., a calf, and compress the portion of the person's leg to stimulate blood flow in the leg. A compression device may include a motor to deliver air through tubing to inflatable chambers in the sleeve, which compresses the portion of the person's leg as the inflatable chambers fill with air. The motor can produce high noise levels, which may cause discomfort for the person using the compression device. Additionally, the sleeves can trap heat around the portion of the person's leg, which may also cause discomfort for the person using the compression device. Furthermore, because the person is tethered to the compression device, the tubing of the compression device may become tangled (e.g., increasing a risk that the person may fall) or bound (e.g., preventing the sleeves from compressing the muscle tissue as intended) if the person moves from a lying position while wearing the sleeves. In other words, a person that may otherwise be able to move, e.g., to a sitting position, a standing position, etc., or walk, e.g., for brief durations, may be required to remain in the lying position while using the compression devices. As such, a need has arisen to better treat VT and DVT.

### BRIEF DESCRIPTION OF THE DRAWINGS

FIG. 1 is a perspective view of an example medical device on a person's leg.

FIG. 2 is an exploded view of the device inclusive of a housing and a pad.

FIG. 3 is a perspective view of a conductive pad connected with wires.

FIG. 4 is a block diagram of an example system for the device.

FIGS. 5A-5C are plots of example waveforms for an electrical signal provided to stimulate blood flow in a person's muscle tissue.

FIG. 6A is a first part of a flowchart of an example process for optimizing the electrical signal provided to stimulate blood flow in a person's muscle tissue.

FIG. 6B is a second part of the flowchart of FIG. 6A.

### DETAILED DESCRIPTION

With reference to the Figures, a device 10 includes a conductive pad 16, an electrode 14 in electrical communication with the conductive pad 16, and a controller 20. The controller 20 is programmed to provide a plurality of temporally successive electrical signals to the electrode 14; determine a maximum amount of muscle contraction in response to the provided electrical signals based on sensor data; and select an optimized electrical signal by determining a minimum voltage that corresponds to the maximum amount of muscle contraction.

Typically, compression devices such as SCDs or IPCs are used as a prophylaxis for DVT. The compression devices typically include sleeves that encircle a person's leg and have inflatable chambers that fill with air to stimulate blood flow through the person's leg by compressing muscle tissue in the leg. For example, the compression devices can compress and decompress the muscle tissue to simulate muscle contraction that occurs while the person is walking. However, compression devices typically compress and decompress the muscle tissue in a predetermined manner, e.g., compressing and decompressing during respective time intervals, applying a predetermined compressive force to the muscle tissue, etc. That is, compression devices may operate in a same way for different people. Additionally, compression devices typically do not provide information about an amount of blood flow through the venous system during compression.

Advantageously, the controller 20 can determine an optimized electrical signal that provides sufficient voltage to cause controlled muscle contraction that stimulates blood flow through the venous system in a manner similar to walking. To determine the optimized electrical signal, the controller can receive and analyze sensor data indicating an amount of muscle contraction in response to a plurality of temporally successive electrical signals provided to the conductive pad 16, which is pressed against the person's skin to discharge voltage to the muscle tissue. Based on the sensor data, the controller 20 can estimate blood flow through the venous system for the person and then adjust subsequent electrical signals based on the estimated blood flow. Upon estimating a maximum blood flow, the controller 20 can identify the electrical signal that caused the maximum estimated blood flow as the optimized electrical signal and output the optimized electrical signal to stimulate muscle contraction in the person's leg that occurs during walking. Using the sensor data that indicates an amount of muscle contraction in response to respective electrical signals allows the controller 20 to determine respective optimized electrical signals for different people, which can increase a likelihood of the device 10 stimulating maximum blood flow through the venous system of different people. By stimulating maximum blood flow through the venous system of a person, the device 10 can be used as a prophylaxis for various medical conditions (e.g., including, but not limited to, DVT, PE, peripheral artery disease, nitric oxide generation, mitigating nerve damage, and osteoblast stimulation for bone growth) and/or for wound healing.

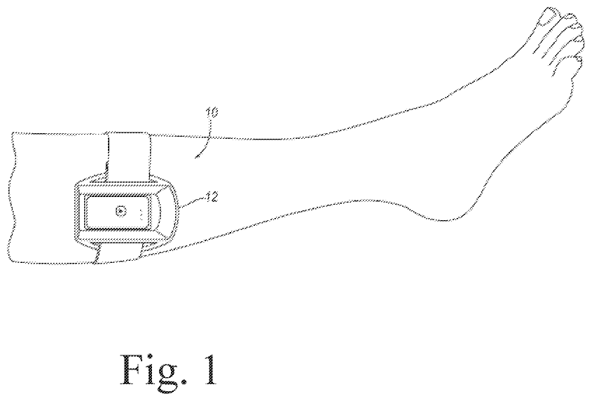

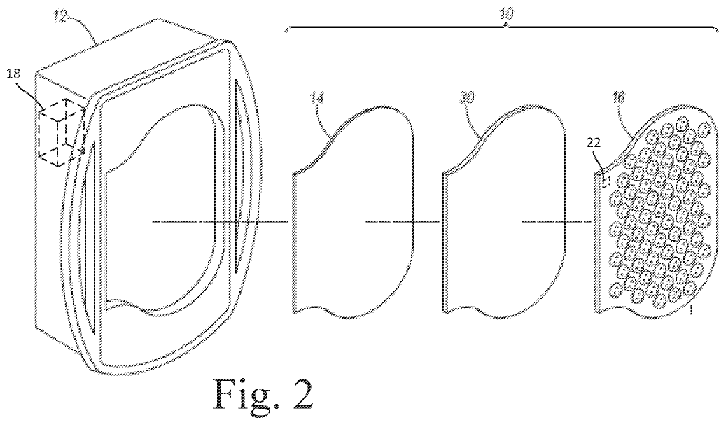

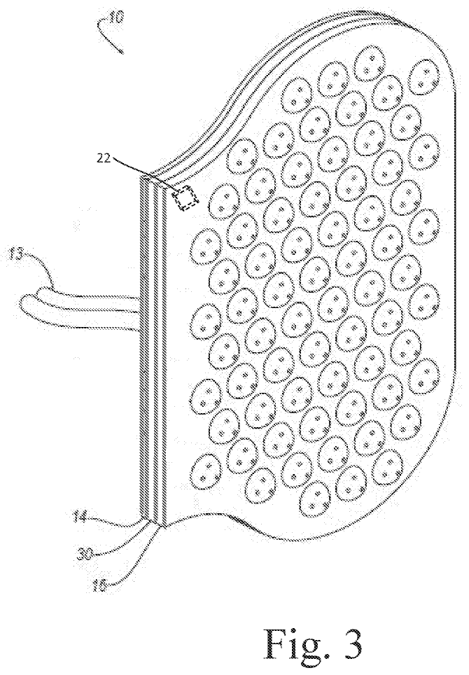

With reference to FIG. 1, the device 10 may be affixed to a person so as to rest against the person's leg, and specifically a calf, to cause controlled muscle contractions that stimulates blood through the venous system. Specifically, the conductive pad 16 may press against the person's calf to transfer an electrical signal to the muscle tissue. In one non-limiting example, the conductive pad 16 may be received in a housing 12, as shown in FIGS. 1 and 2, and in another example, the conductive pad 16 may be directly connected to a power source via wires 13, as shown in FIG. 3. In either example, the device 10 may be held against the person's leg via adhesives, straps, etc.

With reference to FIG. 2, the housing 12 may house various components. For example, the housing 12 may house the controller 20, a power supply 18, a communication module, etc. The housing 12 may be of any suitable type and construction, e.g., a polymer, a composite, a combination of rigid materials, etc. The housing 12 may include a human-machine interface (HMI) (not numbered). The HMI includes user input devices such as buttons, switches, touchscreens, etc. The input devices may include sensors to detect user inputs. The HMI may further include output devices such as displays (including touchscreen displays), lights, etc., that output signals or data to the user. The housing 12 and the conductive pad 16 may move together as a unit.

The power supply 18 provides electricity to various components of the device 10. For example, the power supply 18 can provide electricity to components housed in the housing 12, e.g., the controller 20, the communication module, etc. Additionally, the power supply 18 can provide electricity to the electrode 14. The power supply 18 can include one or more batteries, e.g., 12-volt lithium-ion batteries, and/or one or more power networks to supply power from the batteries to the components. Since the power supply 18 is housed in the housing 12, the conductive pad 16 may lack wires extending from the conductive pad 16 to an external power supply.

The housing 12 may be selectively electrically connectable to a base station (not shown). When the housing 12 is electrically connected to the base station, the base station may transfer electrical energy to the housing 12, e.g., to charge the power supply 18. The power supply 18 may store the electrical energy obtained from the base station. That is, the base station may charge the power supply 18 of the housing 12. The base station and the housing 12 may 12 may each include electrical contacts of a common type such that the electrical contacts can engage each other and transfer electrical energy from the base station to the housing 12. The electrical contacts may take any suitable form, e.g., metal plates, conductive strips, electrical connectors, etc.

The base station may include a second HMI (not shown). The second HMI includes user input devices such as buttons, switches, touchscreens, etc. The input devices may include sensors to detect user inputs. The second HMI may further include output devices such as displays (including touchscreen displays), lights, etc., that output signals or data to the user.

The base station may include a computer (not shown) and a communications module (not shown). The computer can be a conventional computing device, i.e., including one or more processors and one or more memories, programmed to provide operations such as disclosed herein.

The computer may be configured for communicating via the communication module (not shown) with computing devices outside of the base station, e.g., the controller 20, a remote computer, a tablet, a mobile phone, etc. The communications module could include one or more of various wired or wireless communication mechanisms, including any desired combination of wired (e.g., cable and fiber) and/or wireless (e.g., cellular, wireless, satellite, microwave, and radio frequency) communication mechanisms and any desired network topology (or topologies when multiple communication mechanisms are utilized). Example communication networks include wireless communication networks (e.g., using Bluetooth®, Bluetooth® Low Energy (BLE), IEEE 802.11, etc.)

The computer may be programmed to receive a user input via the second HMI. The user input may specify an electrical signal, a person's biometric information (e.g., height, weight, etc.), or any other suitable information. Upon receiving the user input, the computer may provide the user input to the controller 20. For example, the computer may transmit the user input to the controller 20.

It is understood that the housing 12 is optional, and the conductive pad 16 may be attached to the person without a housing 12. For example, the conductive pad 16 may be directly connected to the power supply 18 via wires 13, as shown in FIG. 3, although a wireless approach is preferred. In such an example, the base station may house the various components of the housing 12, such as the controller 20, the power supply 18, etc. The wires 13 may extend from the electrode 14 to the base station, e.g., the controller 20.

The electrode 14 is positioned to distribute an electrical signal to the conductive pad 16. That is, the electrode 14 is disposed between the power supply 18 and the conductive pad 16. In an example in which the device 10 includes the housing 12, the electrode 14 is received in the housing 12, as shown in FIG. 2. In such an example, the electrode 14 is disposed between the housing 12 and the conductive pad 16.

The electrode 14 is in electrical communication with the power supply 18 (see FIG. 3). That is, the electrode 14 receives electricity from the power supply 18. Specifically, the electrode 14 receives voltage from the power supply 18. The electrode 14 may be configured to distribute the voltage substantially uniformly across the electrode 14. For example, the electrode 14 may be formed of any suitable material that distributes electricity substantially uniformly throughout the material. As another example, the electrode 14 may have any suitable thickness that allows electricity to be substantially uniformly distributed throughout the electrode 14. The electrode 14 may be of any suitable type and construction, e.g., a conductive polymer, a metal film, a compound film such as titanium nitride, etc. The electrode 14 preferably has a same size, e.g., a same length and width, and shape as the conductive pad 16. Returning to FIG. 1, the conductive pad 16 may be affixed to a person so as to rest against a person's calf and to cause controlled muscle contractions that stimulates blood flow in the venous system, as set forth above. The conductive pad 16 may be any suitable electrically conductive material, e.g., carbon electrical conductive silicone, and may include any suitable electrically conductive coating, e.g., silver.

The conductive pad 16 may include one or more sensors 22 that can provide data to the controller 20. The data may indicate a mechanomyogram (MMG) of muscle tissue, e.g., in the calf. An MMG is a mechanical signal observable from the person's skin when the muscle tissue is contracted, i.e., an amount of muscle contraction. Additionally, the data can indicate an orientation of the sensor(s) 22, e.g., substantially vertical, substantially horizontal, etc. For example, the sensor(s) 22 can include accelerometers such as microelectromechanical systems (MEMS). It is understood that the conductive pad 16 may include any other suitable sensors 22.

The conductive pad 16 may be affixed to a backing 30, e.g., via electrically conductive double-sided tape. It is understood that the conductive pad 16 may be affixed to the backing 30 in any other suitable manner.

The backing 30 is disposed between the conductive pad 16 and the electrode 14. The backing 30 may be removably attached to the electrode 14. That is, the backing 30 (and the conductive pad 16) may be removed from the electrode 14. For example, the backing 30 (and the conductive pad 16) may be removed from the electrode 14 and replaced with a new backing 30 and conductive pad 16. The backing 30 may be removably attached to the electrode 14 in any suitable manner that conducts electricity, e.g., via a conductive tape, a conductive adhesive, etc. In a preferred embodiment, the backing 30 is flexible conductive silicone rubber. It is understood that the backing 30 may be any other suitable conductive material.

The backing 30 may be a same size and shape as the conductive pad 16. In other words, the backing 30, the electrode 14, and the conductive pad 16 may all be a same size and shape. In this situation, the electrode 14 may distribute voltage substantially uniformly through the backing 30 to the conductive pad 16. In other words, the voltage may be substantially uniformly distributed throughout the conductive pad 16.

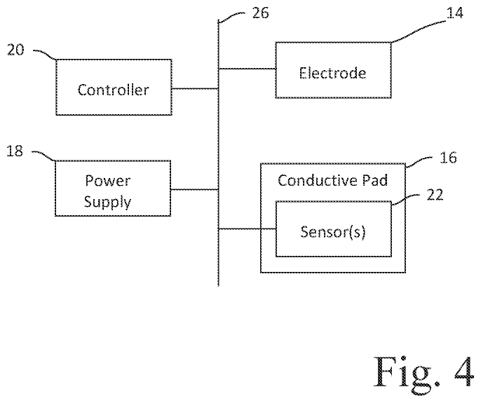

Turning now to FIG. 4, the controller 20 may be a microprocessor-based computing device implemented via circuits, chips, or other electronic components. The controller 20 may 20 may include a processor, memory, etc. The memory of the controller 20 may store instructions executable by the processor and the processor may read the instructions from the memory and execute the instructions.

The controller may be configured for communicating via a communication module (not shown) with computing devices outside of the housing. For example, the controller can be accessed via the network. That is, the controller can provide data to and/or receive data from the computer via the network. The communications module of the housing typically has features in common with the communications module of the base station, and therefore will not be described further to avoid redundancy.

Additionally, the controller 20 may be communicatively coupled to the sensor(s) 22 of the conductive pad 16. The controller 20 is generally arranged for communications on a communication network 26 that can include wired and/or wireless mechanism, e.g., a communications bus. The controller 20 may transmit messages to the sensor(s) 22 and/or receive message from the sensor(s) 22 via the communication network 26. That is, the sensor(s) 22 is(are) in communication with the controller 20 via the communication network 26. For example, the sensor(s) 22 can provide signals, e.g., indicating an MMG of muscle tissue and/or an orientation of the sensor, to the controller 20 via the communication network 26.

The controller 20 may be programmed to actuate various components, e.g., the power supply 18, to provide an electrical signal to the electrode 14. As set forth above, the electrode 14 distributes the electrical signal to the conductive pad 16 such that the electrical signal is transferred to the person's calf. The controller 20 may determine parameters for the electrical signal. As used herein, a “parameter” is a value of a measurement of a physical characteristic of the electrical signal. Non-limiting examples of parameters include an amplitude, i.e., a maximum voltage; a frequency; a wavelength; etc. The controller 20 can actuate the various components to output various electrical signals, i.e., various waveforms having various amplitudes and frequencies.

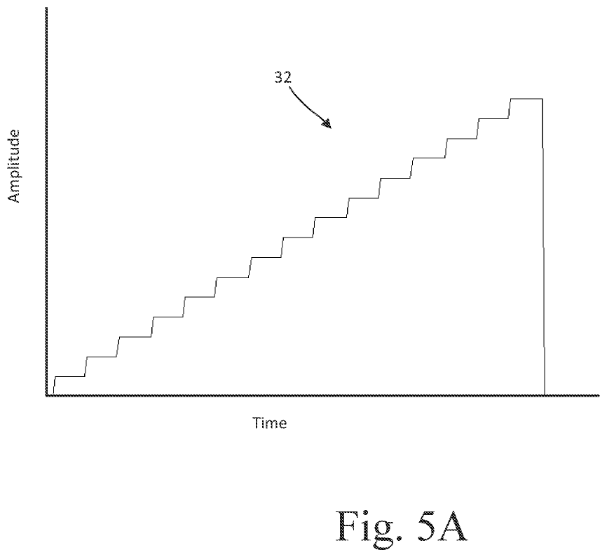

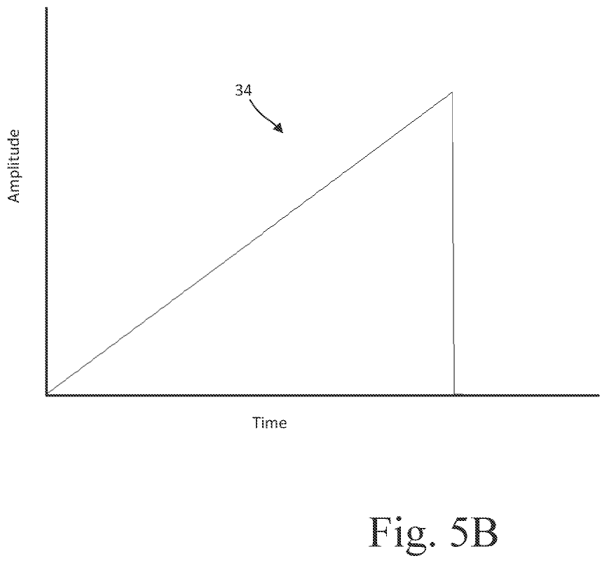

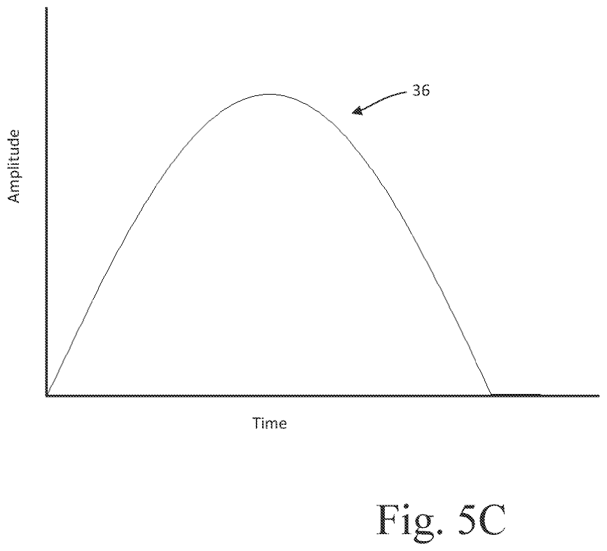

The electrical signal has a waveform, i.e., a curve showing a voltage of the electrical signal over time. The waveform is defined by the parameters for the electrical signal. As one non-limiting example, the electrical signal may be a step wave 32 (see FIG. 5A). As another non-limiting example, the electrical signal may be a sawtooth wave 34 (see FIG. 5B). As another non-limiting example, the electrical signal may be a portion of a sine wave 36 (see FIG. 5C). It is to be understood that the electrical signal may have any other suitable waveform, e.g., square, triangle, rectangle, etc.

The waveform includes a stimulation period and a rest period. During the stimulation period, voltage is being applied to the person's calf, which may cause muscle contraction of the calf. That is, the voltage of the electrical signal may be non-zero during the stimulation period of the waveform. During the rest period, no voltage is being applied to the person's calf, which may allow muscle relaxation to occur. That is, the voltage of the electrical signal may be zero during the rest period of the waveform.

The stimulation period may have a duration between 0.5 seconds and 3 seconds, inclusive. Preferably, the stimulation period may have a duration of 1.8 seconds. It is to be understood that the stimulation period may have other durations.

The rest period may have a same or different duration than the stimulation period. For example, the stimulation period and the rest period may have different durations. That is, the electrical signal may be an asymmetrical waveform, i.e., a pulse waveform (as shown in FIGS. 5A-B). As one non-limiting example, the rest period may be shorter than the stimulation period. It is to be understood that the electrical signal may be a symmetrical waveform (see FIG. 5C), i.e., the stimulation period and the rest period may have a same duration.

The controller 20 may be programmed to determine an electrical signal that induces blood flow through the venous system. Blood flow is an amount of blood moving through the venous system per unit time, e.g., measured over a predetermined time period, e.g., 30 seconds, one minute, etc. The controller 20 can estimate the blood flow through the venous system based on muscle contraction of the calf. As one example, upon determining an MMG of the muscle tissue in the calf in response to an electrical signal, the controller 20 can multiply the MMG by a calibration constant that correlates muscle contraction to blood flow through the venous system. The calibration constant can be determined, e.g., based on empirical testing that determines blood flow, e.g., volume, rate, etc., through respective venous systems of different people in response to various amounts of muscle contraction, i.e., various MMGs, of the respective calves. The controller 20 can then estimate the blood flow based on a number of muscle contractions within the predetermined time and the product of the MMG and the calibration constant. The controller 20 may increment a counter specifying the number of muscle contractions upon receiving an MMG via the sensor data. The controller 20 may then reset the counter after the predetermined time.

Additionally, or alternatively, the controller 20 can estimate the blood flow through the venous system based on relaxation of the muscle tissue in the calf. In such an example, the controller 20 can determine, e.g., based on sensor data, an amount of time for the muscle tissue in the calf to fully relax and can determine the blood flow based on the amount of time and a vascular refill for the muscle tissue in the calf. The controller 20 can, for example, determine the amount of time for the muscle tissue in the calf to fully relax based on the MMG of the muscle tissue in the calf. The vascular refill for the muscle tissue specifies an amount of time for blood to refill a fully contracted muscle. The vascular refill for muscle tissue can be determined, e.g., based on empirical testing that determines respective amounts of times for muscle tissue in calves of different people to refill from fully contracted states.

To determine the electrical signal that maximizes blood flow through the venous system, the controller 20 outputs a plurality of temporally successive electrical signals. The controller 20 may be programmed to determine parameters, e.g., a wavelength, an amplitude, a frequency, etc., for an initial electrical signal. For example, the parameters for the initial electrical signal may be predetermined, e.g., stored in a memory of the controller 20. In this situation, the controller 20 can access the memory to retrieve the parameters for the initial electrical signal. As another example, the memory of the controller 20 may store a plurality of electrical signals specifying respective parameters, e.g., amplitude, frequency, waveform, etc. In the situation, the controller 20 can, for example, determine the parameters for the initial electrical signal based on a user input to the HMI (or second HMI) selecting a corresponding stored electrical signal. The controller 20 can then access the memory to retrieve the parameters for the selected electrical signal.

Upon determining the parameters for the initial electrical signal, the controller 20 can actuate the various components to output the initial electrical signal according to the determined parameters. That is, the controller 20 can actuate the various components to provide voltage to the electrode according to a waveform that satisfies the parameters. Upon outputting the initial electrical signal, the controller 20 receives, from the sensor(s) 22, data indicating an MMG of the muscle tissue in the calf and an orientation of the sensor 22 during the stimulation period of the electrical signal. The controller 20 can receive data from the sensor(s) 22 at various sampling rates. As one non-limiting example, the sampling rates can vary between 12.5 Hz and 200 Hz, inclusive. For example, the sampling rate may be 50 Hz. It is to be understood that the controller 20 may receive data from the sensor(s) 22 at other sampling rates. The controller 20 can store the received data, e.g., in a buffer, memory, etc., during the stimulation period.

During the rest period, the controller 20 can analyze the received data. The controller 20 can determine a physical state of the person, i.e., a position of the person, e.g., standing, reclining, lying down, etc., based on the orientation of the sensor 22. The controller 20 can then compare the determined physical state to a desired physical state, e.g., stored in the memory of the controller 20. The desired physical state specifies one or more physical states for the person that enables the controller 20 to output an electrical signal to the conductive pad 16. For example, a desired physical state may be, e.g., lying down, reclining, sitting, etc. An undesired physical state is standing (or walking). The controller 20 is typically prevented from outputting an electrical signal to the conductive pad 16 when the physical state for the person is an undesired state, e.g., to avoid causing muscle contraction while a person is walking. As one non-limiting example, the controller 20 can determine the person is standing (or walking) based on the orientation of the sensor 22 being substantially vertical. As another non-limiting example, the controller 20 can determine the person is lying down based on the orientation of the sensor 22 being substantially horizontal.

Additionally or alternatively, the controller 20 may be programmed to determine that a component of the device 10 is not operating as desired, e.g., the conductive pad 16 is not properly applied to the person's calf, there is a short circuit between the power supply 18 and the electrode 14, etc., based on the received sensor data. In this situation, the controller 20 may generate an error code that indicates the component and/or the undesired operation. The controller 20 may actuate the HMI to output, e.g., display, the error code. Upon determining that the component of the device 10 is not operating as desired, the controller 20 may be prevented from outputting subsequent electrical signals.

Upon determining that the physical state of the person matches a desired state, the controller 20 can determine a subsequent electrical signal using a machine learning program. The machine learning algorithm is trained to accept the MMG and the electrical signal as input and to output one or more updated parameter(s) for the subsequent electrical signal. As one non-limiting example, the machine learning program may output an updated amplitude for the subsequent electrical signal. The updated amplitude may be determined by increasing or decreasing the amplitude of the initial electrical signal. It is understood that the machine learning program can output additional, or alternative, updated parameters, e.g., frequency, wavelength, etc., that are determined by increasing or decreasing the corresponding parameter of the initial electrical signal. The machine learning algorithm can be trained on sample biometric data for various people and MMG data corresponding to known parameter changes. The machine learning algorithm may be any suitable type, e.g., linear regression, artificial neural network, etc.

The controller 20 can then determine an updated waveform for the subsequent electrical signal based on the updated parameter(s). As one non-limiting example, the initial electrical signal can be scaled up, e.g., the waveform for the initial electrical signal can be multiplied by a constant, or scaled down, e.g., the waveform for the initial electrical signal can be divided by a constant, to generate the updated waveform for the subsequent electrical signal. Upon determining the updated parameter(s) and corresponding waveform for the subsequent electrical signal, the controller 20 is programmed to actuate the various components to output the subsequent electrical signal. Additionally, the controller 20 resets the buffer, memory, etc., e.g., by removing the data received from the stimulation period of the initial (or previous) electrical signal.

Upon outputting the subsequent electrical signal, the controller 20 receives, from the sensor 22, data indicating the MMG of the muscle tissue in the calf and the orientation of the sensor 22. The controller 20 can determine whether to not to output subsequent electrical signals based on the orientation of the sensor 22, e.g., in substantially the same manner as discussed above. Upon determining the physical state of the person is a desired state, as discussed above, the controller 20 can estimate the subsequent blood flow through the venous system based on the MMG of the muscle tissue in response to the subsequent electrical signal, e.g., in substantially the same manner as discussed above. The controller 20 can then compare the subsequent blood flow through the venous system to the initial blood flow through the venous system, i.e., the blood flow estimated in response to the initial electrical signal. If the subsequent estimated blood flow is greater than the initial estimated blood flow, then the controller 20 can determine updated parameters for another subsequent electrical signal, e.g., in substantially the same manner as discussed above. The controller 20 can continue to determine updated parameters for subsequent electrical signals in this manner until the subsequent estimated blood flow is equal to the previous estimated blood flow, i.e., the estimated blood flow corresponding to a previous electrical signal. If the subsequent estimated blood flow is equal to the initial (or previous) estimated blood flow, then the controller 20 can identify maximum estimated blood flow.

Upon determining a maximum estimated blood flow, the controller 20 can identify an optimized electrical signal by selecting the electrical signal with a minimum amplitude, i.e., voltage, that corresponds to the maximum estimated blood flow, i.e., results in the maximum estimated blood flow when the electrical signal is applied to the person's calf, which allows the controller 20 to output an electrical signal that causes controlled muscle contraction to stimulate blood flow through the venous system in a manner similar to walking. A typical amplitude for an optimized electrical signal, e.g., given across skin stimulation with an impedance of approximately 1000 ohms, is between 20V at high frequencies (e.g., 10,000 Hz) and 200 V for low frequencies (e.g., 2 Hz).

The controller 20 may be programmed to then actuate various components to output the optimized signal. It is understood that the housing 12 and/or the base station may receive a user input that specifies an adjustment to the optimized electrical signal. In an example in which the controller 20 receives the user input specifying an adjusted electrical signal, the controller 20 may be programmed to output the adjusted electrical signal. That is, the user input may override the optimized electrical signal determined by the controller 20.

Upon outputting the optimized electrical signal, the controller 20 can continue to receive sensor data indicating an MMG of the muscle tissue and the orientation of the sensor(s) 22, as discussed above. The controller 20 can continue to monitor the orientation of the sensor 22, e.g., in substantially the same manner as discussed above, to prevent outputting subsequent optimized electrical signals upon detecting the person is in an undesired physical state. Additionally, or alternatively, the controller 20 may be programmed to determine a subsequent optimized electrical signal, e.g., in substantially the same manner as discussed above, based on, e.g., detecting a change in the MMG of the muscle tissue, after a predetermined time period, etc.

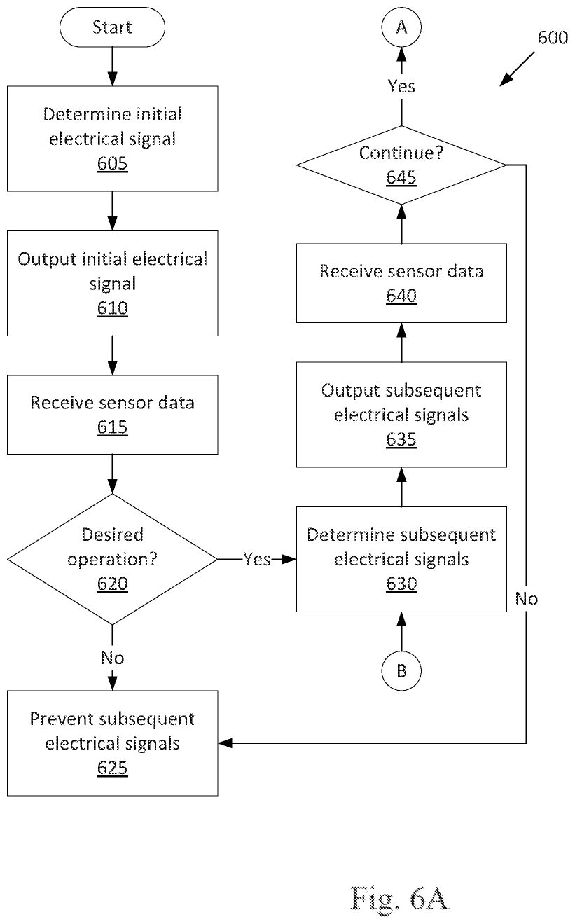

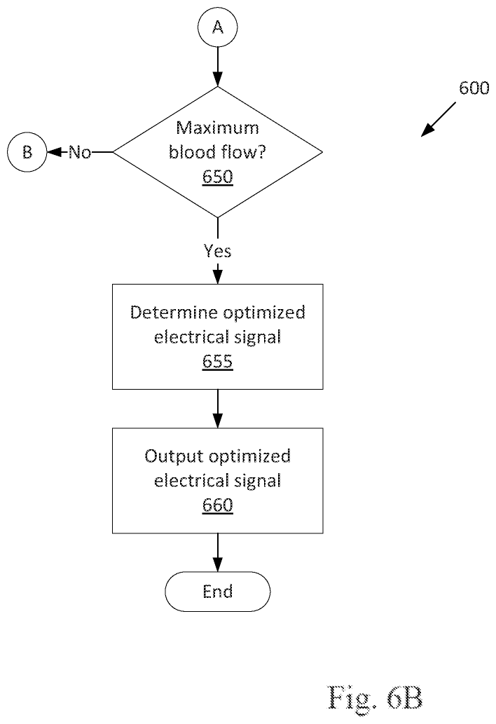

With reference to FIGS. 6A-B, the controller 20 stores instructions to determine an electrical signal that maximizes blood flow through the venous system according to a process 600. Specifically, the controller 20 outputs a plurality of temporally successive electrical signals having various waveforms and determines an MMG for the muscle tissue corresponding to the respective electrical signals. Upon estimating a maximum blood flow through the venous system based on the MMGs corresponding to respective electrical signals, the controller 20 selects an electrical signal having a minimum voltage that corresponds to the maximum estimated blood flow, which allows the controller 20 to output an electrical signal that causes controlled muscle contraction to stimulate blood flow through the peroneal artery in a manner similar to walking.

The process 600 begins in a block 605, as shown in FIG. 6A. In the block 605, the controller 20 determines parameters, e.g., a wavelength, an amplitude, and a frequency, of an initial electrical signal. The controller 20 can access a memory to retrieve the parameters for the initial electrical signal, as discussed above. The initial electrical signal includes a stimulation period and a rest period, as discussed above. The process 600 continues in a block 610.

In the block 610, the controller 20 actuates various components, e.g., the power supply 18, to output the initial electrical signal. That is, the controller 20 actuates the various components to output voltage according to a waveform that satisfies the parameters determined in the block 605. The process 600 continues in a block 615.

In the block 615, the controller 20 receives data from the sensor(s) 22 during the stimulation period, i.e., while voltage is applied to the person's calf. The data indicates an MMG for the muscle tissue in the calf and an orientation of the sensor 22, as discussed above. The process continues in a decision block 620.

In the decision block 620, the controller 20 determines whether to determine parameters for a subsequent electrical signal. The controller 20 can determine a physical state of the person based on the orientation of the sensor 22, as discussed above. The controller 20 can then compare the determined physical state to a stored desired physical state, as discussed above. Additionally, or alternatively, the controller 20 may be programmed to determine whether the components of the device 10 are operating as desired, as discussed above. If the controller 20 determines that the determined physical state does not match a desired physical state or that at least one component is not operating as desired, then the process 600 continues in a block 625. If the controller 20 determines that the determined physical state matches a desired physical state and the components are operating as desired, then the process 600 continues in a block 630.

In the block 625, the controller 20 determines to not output a subsequent electrical signal. That is, the controller 20 prevents the power supply 18 from outputting an electrical signal while an error code is present or the person is in an undesired physical state. Once the error code is cleared or the person returns to the desired physical state, the process 600 returns to the block 605.

In the block 630, the controller 20 determines updated parameters for a subsequent electrical signal. For example, the controller 20 can input the MMG and the electrical signal into a machine learning algorithm that outputs the updated parameters for the subsequent electrical signal, as discussed above. The process 600 continues in a block 635.

In the block 635, the controller 20 outputs the subsequent electrical signal. The block 635 is substantially identical to the block 610 and will therefore not be described further to avoid redundancy. The process 600 continues in a block 640.

In the block 640, the controller 20 receives data from the sensor(s) 22 during the stimulation period, i.e., while voltage is applied to the person's calf. The block 640 is substantially identical to the block 615 and will therefore not be described further to avoid redundancy. The process 600 continues in a block 645.

In the block 645, the controller 20 determines whether to continue. The block 645 is substantially identical to the block 620 and will therefore not be described further to avoid redundancy. If the controller 20 determines to continue, then the process 600 continues in a block 650. Otherwise, the process 600 continues in the block 625.

Turning now to FIG. 6B, following the block 645 shown in FIG. 6A, in the block 650, the controller 20 determines whether the electrical signal stimulated maximum blood flow through the venous system, e.g., based on respective MMGs for successive electrical signals. That is, the controller 20 can estimate blood flow through the venous system based on respective MMGs detected in response to respective electrical signals, as discussed above. The controller 20 then compares the estimated blood flows associated with the respective electrical signals to identify a maximum blood flow, as discussed above. If the controller 20 identifies the maximum blood flow, then the process 600 continues in a block 655. Otherwise, the process 600 returns to the block 630.

In the block 655, the controller 20 determines optimized parameters for an electrical signal. The optimized parameters correspond to an electrical signal having a minimum voltage that results in the maximum blood flow through the venous system, as discussed above. The process 600 continues in a block 660.

In the block 660, the controller 20 can actuate the various components to output a plurality of temporally successive optimized electrical signals. The controller 20 may be programmed to output the plurality of optimized electrical signals for a time period, e.g., stored in a memory of the controller or specified by a user input via the HMI (or second HMI). Alternatively, the controller 20 can actuate the various components to output a plurality of temporally successive electrical signals specified by a user input, as discussed above. The process 600 ends following the block 660.

Computing devices, such as the controller, generally include computer-executable instructions, where the instructions may be executable by one or more computing devices such as those listed above. Computer-executable instructions may be compiled or interpreted from computer programs created using a variety of programming languages and/or technologies, including, without limitation, and either alone or in combination, Java™, C, C++, Visual Basic, Java Script, Perl, etc. Some of these applications may be compiled and executed on a virtual machine, such as the Java Virtual Machine, the Dalvik virtual machine, or the like. In general, a processor (e.g., a microprocessor) receives instructions, e.g., from a memory, a computer-readable medium, etc., and executes these instructions, thereby performing one or more processes, including one or more of the processes described herein. Such instructions and other data may be stored and transmitted using a variety of computer-readable media.

A computer-readable medium (also referred to as a processor-readable medium) includes any non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random access memory (DRAM), which typically constitutes a main memory. Such instructions may be transmitted by one or more transmission media, including coaxial cables, copper wire and fiber optics, including the wires that comprise a system bus coupled to a processor of an ECU. Common forms of computer-readable media include, for example, RAM, a PROM, an EPROM, a FLASH-EEPROM, any other memory chip or cartridge, or any other medium from which a computer can read.

In some examples, system elements may be implemented as computer-readable instructions (e.g., software) on one or more computing devices (e.g., servers, personal computers, etc.), stored on computer readable media associated therewith (e.g., disks, memories, etc.). A computer program product may comprise such instructions stored on computer readable media for carrying out the functions described herein.

The disclosure has been described in an illustrative manner, and it is to be understood that the terminology which has been used is intended to be in the nature of words of description rather than of limitation. Many modifications and variations of the present disclosure are possible in light of the above teachings, and the disclosure may be practiced otherwise than as specifically described.

What is claimed is:

1. A device comprising:
a conductive pad;
an electrode in electrical communication with the conductive pad; and
a controller programmed to:
actuate a power supply to provide a plurality of temporally successive electrical signals to the electrode;
determine a maximum amount of muscle contraction in response to the temporally successive electrical signals based on sensor data; and
select an optimized electrical signal by determining a minimum voltage that corresponds to the maximum amount of muscle contraction.

2. The device of claim 1, further comprising a housing, wherein the electrode is received in the housing.

3. The device of claim 2, wherein the electrode is disposed between the housing and the conductive pad.

4. The device of claim 2, further comprising the power supply, wherein the power supply is housed in the housing.

5. The device of claim 2, wherein the housing houses the controller.

6. The device of claim 1, further comprising a sensor configured to output the sensor data, wherein the conductive pad includes the sensor.

7. The device of claim 1, wherein the electrode and the conductive pad have a same shape.

8. The device of claim 1, further comprising a backing disposed between the conductive pad and the electrode, wherein the backing is removably attached to the electrode.

9. A computer comprising a processor and a memory, the memory storing instructions executable by the processor to:
actuate a power supply to provide a plurality of temporally successive electrical signals to an electrode;
determine a maximum amount of muscle contraction in response to the temporally successive electrical signals based on sensor data; and
select an optimized electrical signal by determining a minimum voltage that corresponds to the maximum amount of muscle contraction.

10. The computer of claim 9, wherein the sensor data includes a mechanomyogram of muscle tissue.

11. The computer of claim 9, wherein the instructions further include instructions to determine a parameter defining a waveform of the optimized electrical signal.

12. The computer of claim 11, wherein the parameter is an amplitude of the waveform.

13. The computer of claim 11, wherein the parameter is an updated parameter, and the instructions to determine the updated parameter include instructions to increase or decrease a first parameter defining a waveform of an initial electrical signal of the temporally successive electrical signals.

14. The computer of claim 9, wherein the instructions further include instructions to determine a parameter defining a waveform of an initial electrical signal of the temporally successive electrical signals by accessing a predetermined parameter from the memory.

15. The computer of claim 9, wherein the instructions further include instructions to determine a parameter defining a waveform of an initial electrical signal of the temporally successive electrical signals based on a user input.

16. The computer of claim 9, wherein the temporally successive electrical signals include a stimulation period and a rest period.

17. The computer of claim 9, wherein the instructions further include instructions to, upon determining that a physical state of a person is an undesired state, prevent the power supply from providing the temporally successive electrical signals to the electrode.

18. The computer of claim 17, wherein the instructions further include instructions to determine the physical state from sensor data from a sensor indicating an orientation of the sensor.

19. The computer of claim 17, wherein the undesired state includes at least one of standing or walking.

20. A method comprising:
actuating a power supply to provide a plurality of temporally successive electrical signals to an electrode;
determining a maximum amount of muscle contraction in response to the temporally successive electrical signals based on sensor data; and
selecting an optimized electrical signal by determining a minimum voltage that corresponds to the maximum amount of muscle contraction.
